(19) United States
(12) Patent Application Publication
(10) Pub. No.: US 2008/0153896 A1
(43) Pub. Date: Jun. 26, 2008

(54) Polymorphic Forms of an HMG-CoA Reductase Inhibitor and Uses Thereof

(76) Inventors: Gyan Chand Yadav, Ghaziabad (IN); Mohammad Baqer, Tahsil-Akbarpur (IN); Vishwesh P. Pandya, Jamnagar (IN)

Correspondence Address: SHERIDAN ROSS PC, 1560 BROADWAY, SUITE 1200, DENVER, CO, 80202, US

(21) Appl. No.: 11/777,503

(22) Filed: Jul. 13, 2007

(30) Foreign Application Priority Data
Jul. 14, 2006 (IN) 1629/DEL/2006

(51) Int. Cl. A61K 31/40; C07D 207/34; A61P 9/10; A61P 9/12; A61P 9/00; A61P 19/10; A61P 25/28

(52) U.S. Cl. 514/423; 548/537

(57) ABSTRACT

The invention provides polymorphic forms of the HMG-CoA reductase inhibitor (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt. The invention also provides methods for preparing these polymorphic forms, pharmaceutical formulations containing these polymorphic forms and methods of using the polymorphic forms of this HMG-CoA reductase inhibitor.

### CROSS REFERENCE TO RELATED APPLICATION

This application claims the benefit of priority under 35 U.S.C. § 119(a) to Indian Patent Application No. 1629/DEL/2006, filed Jul. 14, 2006, which is incorporated herein by reference.

### FIELD OF THE INVENTION

The invention relates to novel forms of the HMG-CoA reductase inhibitor (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid hemi calcium salt. The invention also provides methods for preparing these novel forms, pharmaceutical formulations containing these novel forms and methods of using the novel forms of this HMG-CoA reductase inhibitor.

### BACKGROUND OF THE INVENTION

The compound (3R,5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid hemi calcium salt, having the structure of Formula I has been described in PCT Publication No. WO 2004/106299 (PCT Application No. PCT/IB2004/001761, filed 28 May 2004, incorporated herein by reference).

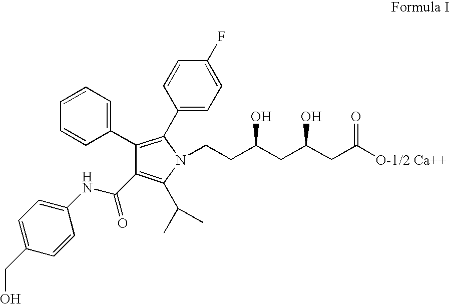

The compound of Formula I has utility in inhibiting 3-hydroxy-3-methylglutaryl-coenzyme A (HMG-CoA), which catalyzes one of the key rate-limiting steps in the biosynthetic pathway of cholesterol formation. Inhibitors of this enzyme are used to treat cardiovascular diseases, including hypercholesterolemia or hyperlipidemia.

The compound of Formula I has been found to possess important attributes, including, (a) it is equipotent to atorvastatin, (b) it is more potent than atorvastatin in inhibiting cholesterol synthesis in an in vivo rat model, (c) the intrinsic clearance of the compound of Formula I in human liver microsomes is significantly less than atorvastatin, (d) it is not a major substrate for the metabolic enzyme CYP3A4 (cytochrome P450 3A4), (e) the compound of Formula I exhibits greater potency and selectivity in the inhibition of cholesterol synthesis in rat primary hepatocytes over inhibition of cholesterol synthesis in extra hepatic cells/cell lines [e.g. NRK-49F (Fibroblast) and L6 (Myoblast)] than does atorvastatin, and (f) it has better hepatoselectivity than does atorvastatin.

One method for producing a compound of Formula I is described in PCT Publication No. WO 2004/106299. Additionally, PCT Publication Nos. WO 2007/054790 and WO 2007/054896 also describe improved and novel processes, respectively, for the preparation of (3R,5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid hemi calcium salt. The product obtained following the processes disclosed in these references is amorphous, and therefore more difficult to use in formulating a pharmaceutical preparation containing this compound, and in producing it on a commercial scale. Additionally, storage of these amorphous compounds for long periods can be problematic.

Therefore, there is a need to produce the hemi calcium salt of 3R,5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid in a reproducible, pure and crystalline form to enable formulations to meet exacting pharmaceutical requirements and specifications. Furthermore, it is economically desirable to produce this compound in a form that is stable for extended periods of time without the need for specialized storage conditions.

### SUMMARY OF THE INVENTION

The present invention provides polymorphic forms of the hemi calcium salt of (3R,5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, which can be used as 3-hydroxy-3-methylglutaryl-coenzyme A (HMG-CoA) reductase inhibitors. The crystalline polymorphs of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid hemi calcium salt referred to as “Form I”, “Form II”, “Form III”, and “Form IV”, which can be used as 3-hydroxy-3-methylglutaryl-coenzyme A (HMG-CoA) reductase inhibitors. The polymorphic forms have a good thermal stability and solubility characteristics and can be characterized by their X-ray diffraction patterns (XRD), infrared spectra (IR) and differential scanning calorimetry (DSC) characteristics.

One embodiment of the present invention is a crystalline polymorph of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated “Form I” and characterized by an X-ray diffraction pattern having peaks at about 5.43, 7.95, 9.61, 11.29, 11.92, 18.91, 19.25, 22.78, and 23.95 degrees two theta. Form I can also be characterized by IR bands at 3301, 2964, 2871, 1902, 1646, 1314, 1225, 1157, 845, 699, 618 and 522 cm⁻¹. Further, Form I can be characterized by a differential scanning calorimetry curve that exhibits an endotherm with an extrapolated onset temperature of about 176.43° C. and associated heat of about 13.55 J/gram.

Also provided herein is a crystalline polymorph of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated “Form II” and characterized by an X-ray diffraction pattern having peaks at about 3.76, 6.08, 7.19, 8.90, 12.30, 12.86, 17.62, 20.16, 24.41, 26.59 and 28.77 degrees two theta. Form II can also be characterized by IR bands at 3398, 2929, 2364, 1738, 1703, 1656, 1596, 1561, 1511, 1314, 1225, 1117, 843, 752 and 700 cm⁻¹. Further, Form II can be characterized by a differential scanning calorimetry curve that exhibits an endotherm with an extrapolated onset temperature of about 187° C. and associated heat of about 21.64 J/gram.

Also provided herein is a crystalline polymorph of (3R,5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated “Form III” and characterized by an X-ray diffraction pattern having peaks at about 4.72, 7.01, 9.38, 13.59, 18.28, 19.56, 20.48, 22.33, 22.97, 23.51 and 27.29 degrees two theta. Form III can also be characterized by IR bands at 3402, 2966, 1655, 1560, 1514, 1222, 1156, 1110, 1031, 844 and 700 cm⁻¹. Further, Form III can be characterized by a differential scanning calorimetry curve that exhibits an endotherm with an extrapolated onset temperature of about 178.49° C. and associated heat of about 18.14 J/gram.

Also provided herein is a crystalline polymorph of (3R,5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated as “Form IV” and characterized by an X-ray diffraction pattern having peaks at about 5.72, 9.42, 10.16, 10.42, 11.40, 18.56, 19.48, 21.03 and 21.83 degrees two theta. Form IV can also be characterized by IR bands at 3400, 2965, 2343, 1650, 1563, 1409, 1013 and 619 cm⁻¹. Further, Form IV can be characterized by a differential scanning calorimetry curve, which exhibits an endotherm with an extrapolated onset temperature of about 179° C. and associated heat of about 11.23 J/gram.

Also provided herein are processes for the preparation of the polymorphic forms of the compounds of Formula I. These processes include preparing a solution of amorphous forms, or any polymorphic forms of the hemi calcium salt of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid including solvates, anhydrous preparations, or preparations in one or more solvents, and then recovering at least one polymorphic form of these compounds from the solution by removing the solvent, and optionally drying the product obtained.

A related embodiment of the present invention is a pharmaceutical composition comprising one or more polymorphic forms of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt. Such pharmaceutical compositions can also include one or more pharmaceutically acceptable carriers, diluents, excipients or mixtures thereof.

These polymorphic forms of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, described herein, and pharmaceutical compositions containing these polymorphic compounds, can be used to treat cholesterol-related diseases, diabetes and related disease states in a mammal, including cerebrovascular diseases and cardiovascular diseases. Specific disease states to be treated by the administration of these polymorphic compounds may include arteriosclerosis, atherosclerosis, hypercholesterolemia, hyperlipidemia, hyperlipoproteinemia, hypertriglyceridemia, hypertension, stroke, ischemia, endothelium dysfunction, peripheral vascular disease, peripheral arterial disease, coronary heart disease, myocardial infarction, cerebral infarction, myocardial microvascular disease, dementia, Alzheimer's disease, osteoporosis, osteopenia, angina, restenosis or combinations of these disease states in a mammal.

### BRIEF DESCRIPTION OF THE DRAWINGS

FIG. 1 is a powder X-ray diffraction (XRD) pattern of Form I of the polymorphic compounds of the present invention.

FIG. 2 is a powder X-ray diffraction (XRD) pattern of Form II of the polymorphic compounds of the present invention.

FIG. 3 is a powder X-ray diffraction (XRD) pattern of Form III of the polymorphic compounds of the present invention.

FIG. 4 is a powder X-ray diffraction (XRD) pattern of Form IV of the polymorphic compounds of the present invention.

FIG. 5 is a differential scanning calorimetry (DSC) curve of Form I of the polymorphic compounds of the present invention.

FIG. 6 is a differential scanning calorimetry (DSC) curve of Form II of the polymorphic compounds of the present invention.

FIG. 7 is a differential scanning calorimetry (DSC) curve of Form III of the polymorphic compounds of the present invention.

FIG. 8 is a differential scanning calorimetry (DSC) curve of Form IV of the polymorphic compounds of the present invention.

FIG. 9 is an infrared absorption (IR) spectrum of Form I of the polymorphic compounds of the present invention.

FIG. 10 is an infrared absorption (IR) spectrum of Form II of the polymorphic compounds of the present invention.

FIG. 11 is an infrared absorption (IR) spectrum of Form III of the polymorphic compounds of the present invention.

FIG. 12 is an infrared absorption (IR) spectrum of Form IV of the polymorphic compounds of the present invention.

FIG. 13 shows chemical structures depicting one step in a process of producing polymorphic compounds of the present invention.

### DETAILED DESCRIPTION OF THE INVENTION

The present invention is drawn to forms of a hemi calcium salt of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid. Such forms can have good thermal stability and/or solubility characteristics, particularly when prepared as a pharmaceutical formulation.

Generally, the invention provides crystalline polymorphic forms of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated as “Form I”, “Form II”, “Form III”, and “Form IV”, which are characterized by their X-ray diffraction (XRD) patterns, infrared spectra (IR) and differential scanning calorimetry (DSC) characteristics presented in the accompanying figures. Processes for the preparation of these polymorphic forms, pharmaceutical compositions containing these forms and methods of treating cholesterol-related disease, diabetes and related disease, cerebrovascular disease or cardiovascular disease are also provided.

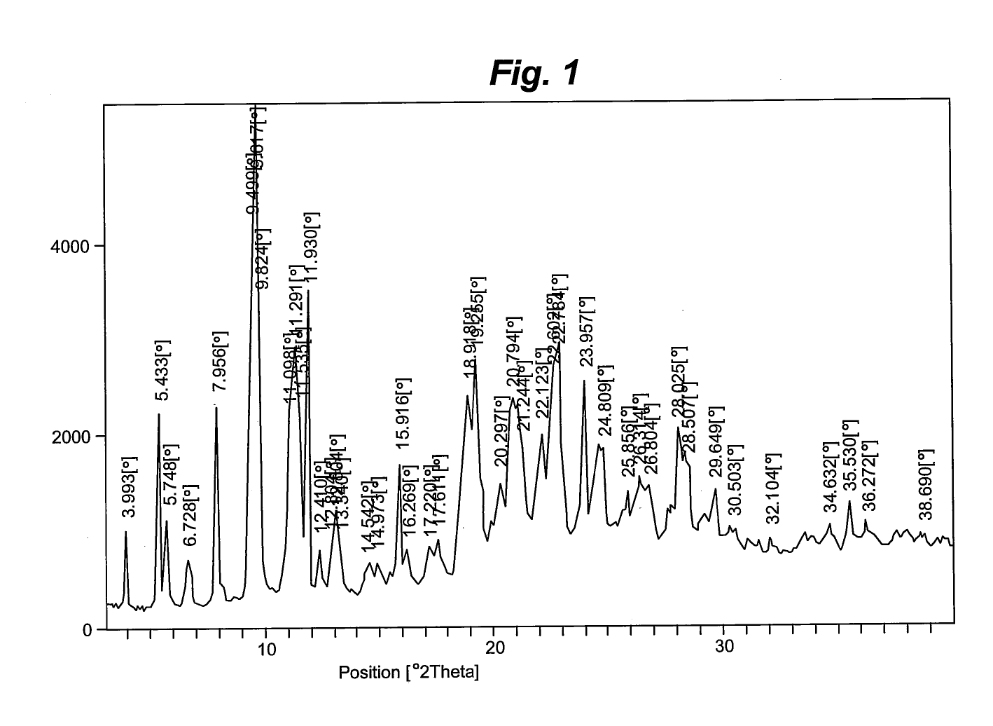

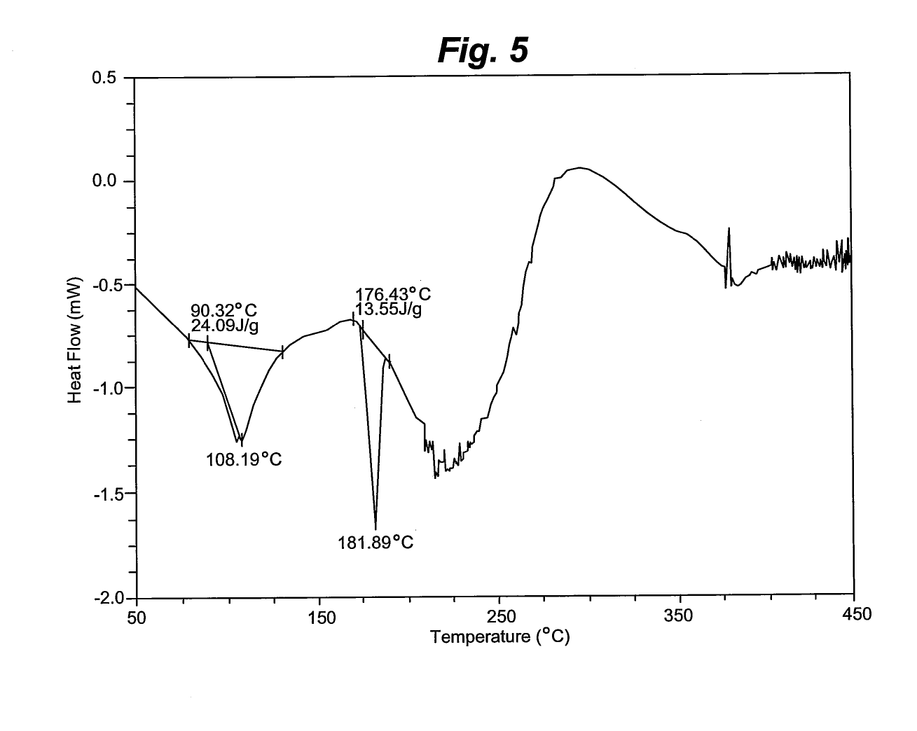

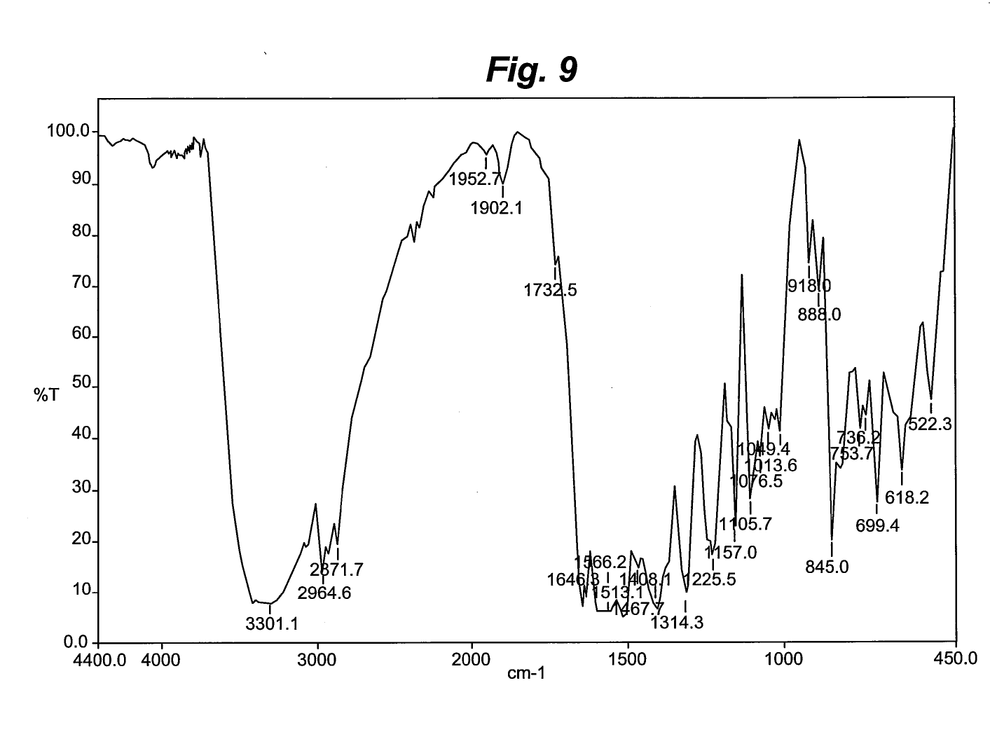

In one aspect, provided herein is a crystalline polymorph of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated “Form I.” Form I may have the X-ray diffraction pattern shown in FIG. 1, the differential scanning calorimetry curve shown in FIG. 5, and the infrared spectrum shown in FIG. 9. The diffraction angles and relative intensities of the X-ray diffraction patterns of Form I are shown in Table 1 (in Example 2). For example, Form I can be characterized by an X-ray diffraction pattern having peaks at about 5.43, 7.95, 9.61, 11.29, 11.92, 18.91, 19.25, 22.78, and 23.95 degrees two theta or by an X-ray diffraction pattern having peaks at about 3.99, 5.43, 5.74, 7.95, 9.61, 11.29, 11.92, 15.91, 18.91, 19.25, 22.78, 23.95, and 28.02° 2θ°. Form I can also be characterized by IR bands at 3301, 2964, 2871, 1902, 1646, 1314, 1225, 1157, 845, 699, 618 and 522 cm⁻¹. Further, Form I can be characterized by a differential scanning calorimetry curve that exhibits an endotherm with an extrapolated onset temperature of about 176.43° C. and associated heat of about 13.55 J/gram.

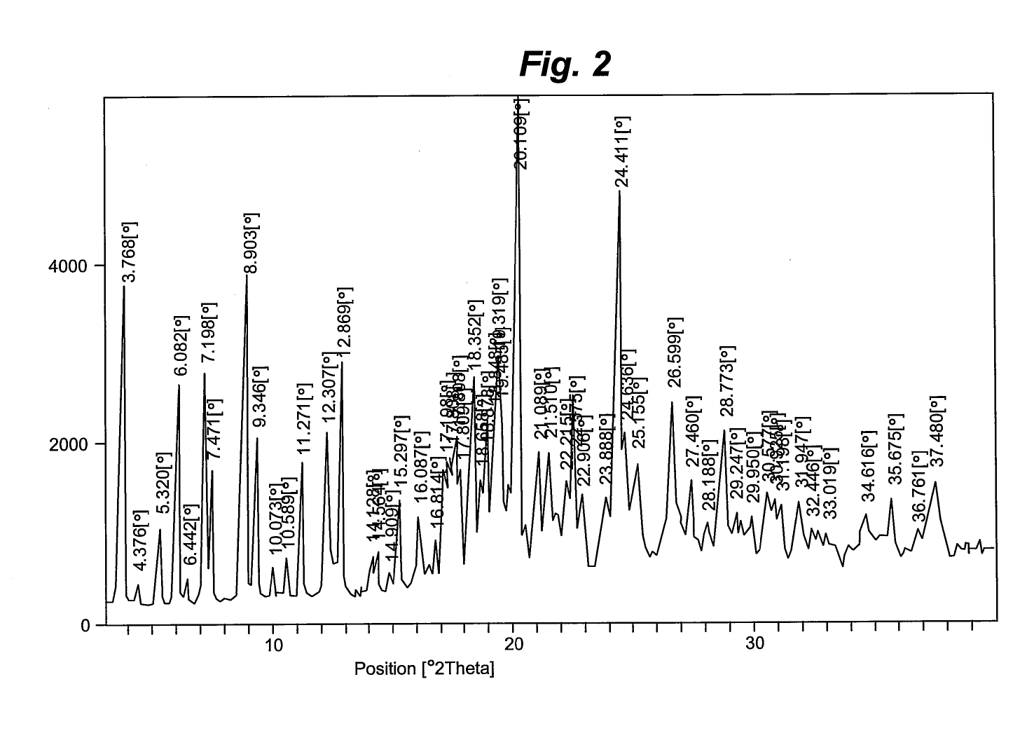

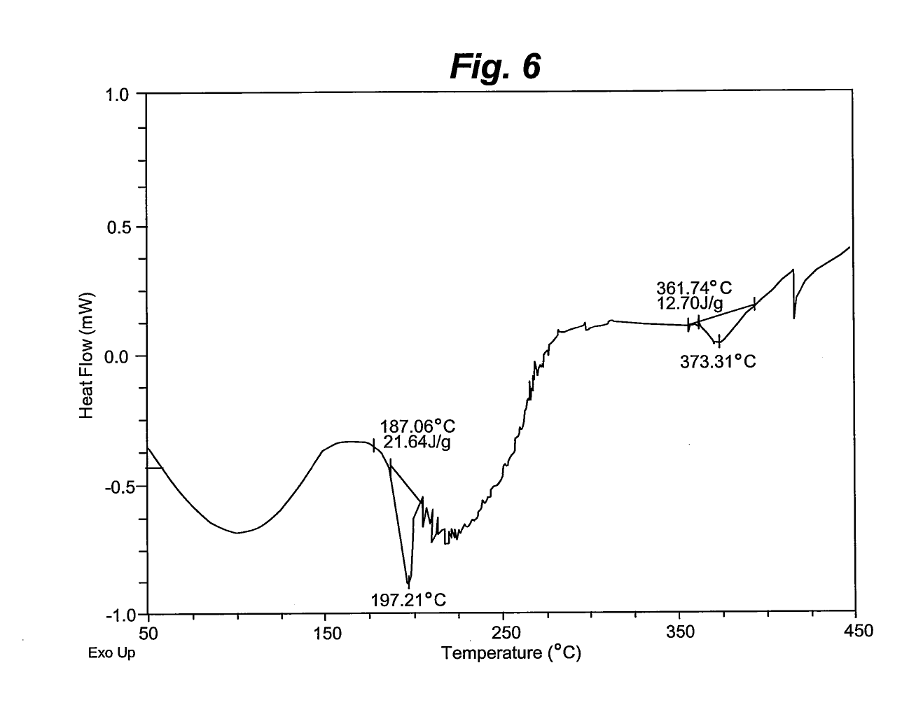

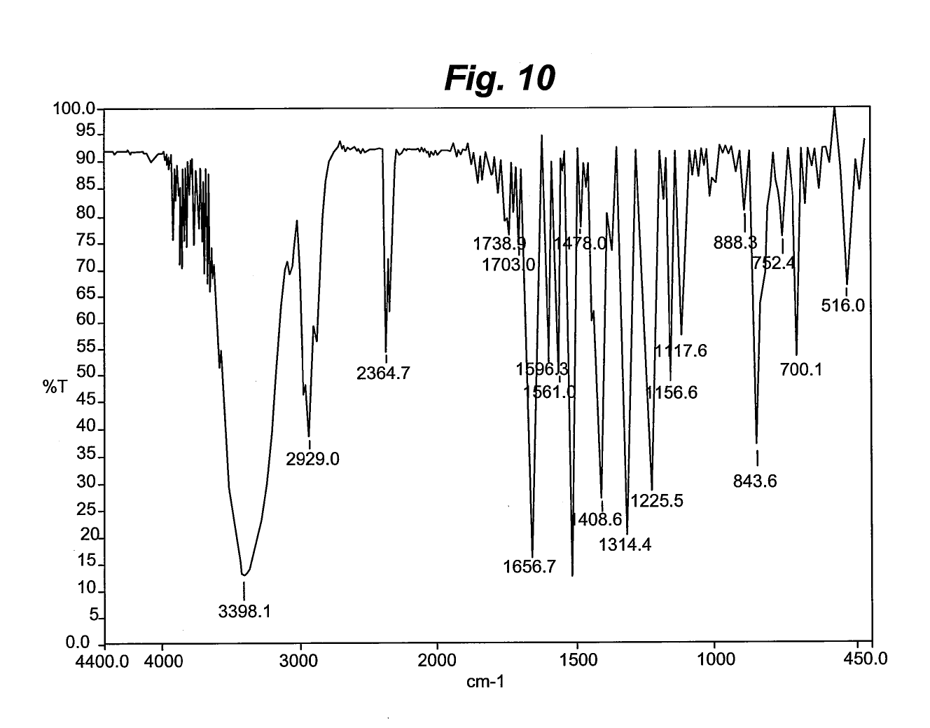

In another aspect, provided herein is a crystalline polymorph of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, designated “Form II.” Form II may have the X-ray diffraction pattern of FIG. 2, the differential scanning calorimetry curve of FIG. 6, and the infrared spectrum of FIG. 10. The diffraction angles and relative intensities of the X-ray diffraction patterns of Form II are shown in Table 2 (in Example 3). For example, Form II can be characterized by an X-ray diffraction pattern having peaks at about 3.76, 6.08, 7.19, 8.90, 12.30, 12.86, 17.62, 20.16, 24.41, 26.59 and 28.77 degrees two theta or by an X-ray diffraction pattern having peaks at about 3.76, 5.32, 6.08, 7.19, 8.90, 9.34, 11.27, 12.30, 12.86, 15.29, 16.18, 17.62, 20.16, 21.08, 21.51, 22.57, 24.41, 24.63, 25.15, 26.59, 28.77, 35.67, 37.48° 2θ°. Form II can also be characterized by IR bands at 3398, 2929,2364, 1738, 1703, 1656, 1596, 1561, 1511, 1314, 1225, 1117, 843, 752 and 700 cm⁻¹. Further, Form II can be characterized by a differential scanning calorimetry curve that exhibits an endotherm with an extrapolated onset temperature of about 187° C. and associated heat of about 21.64 J/gram.

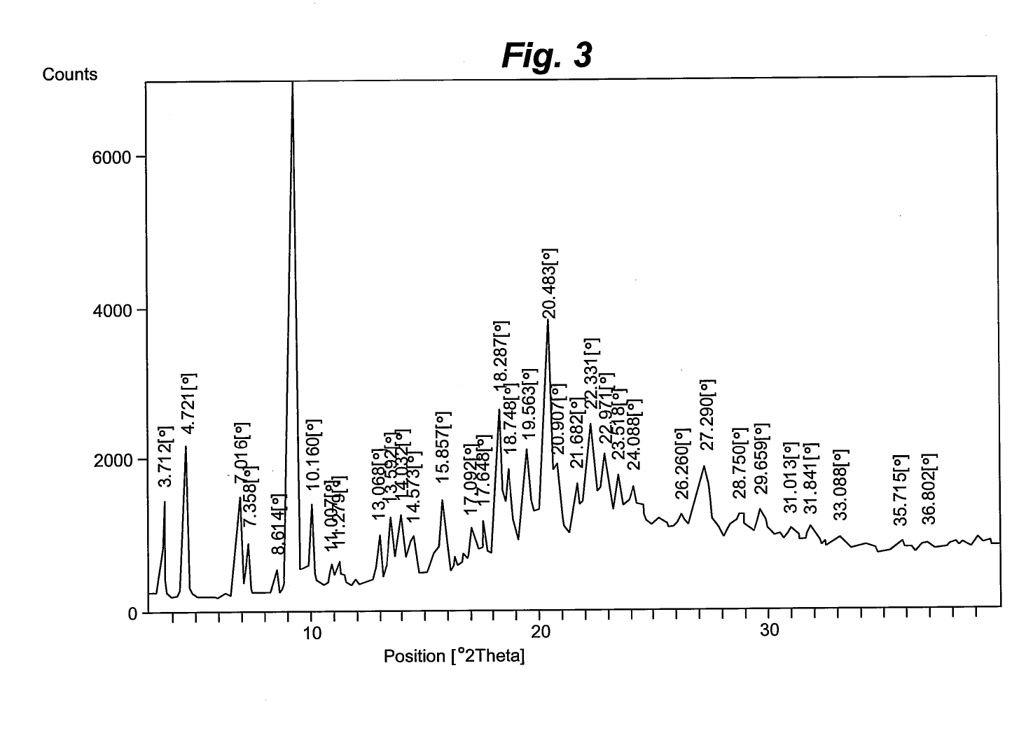

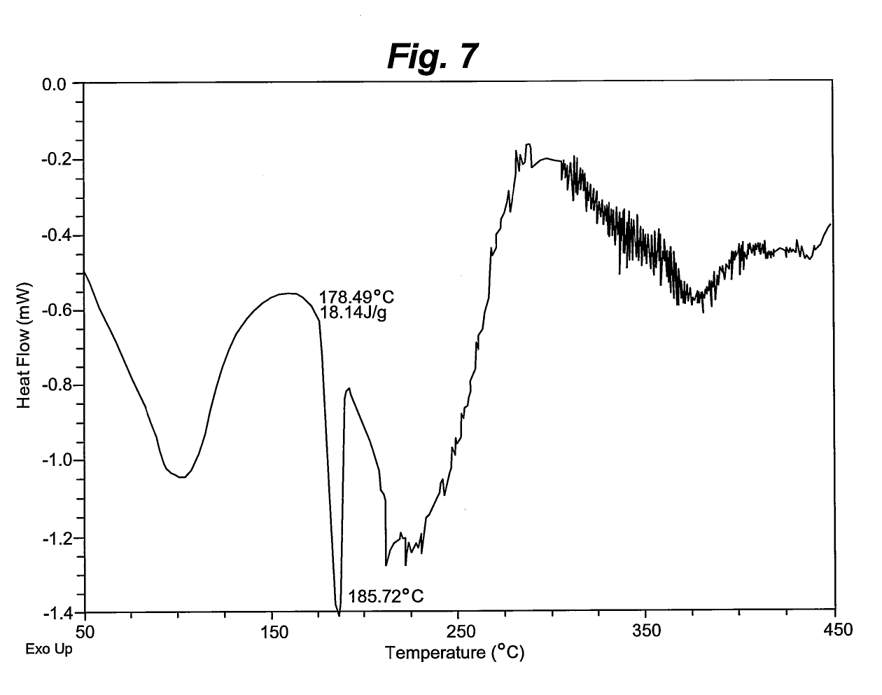

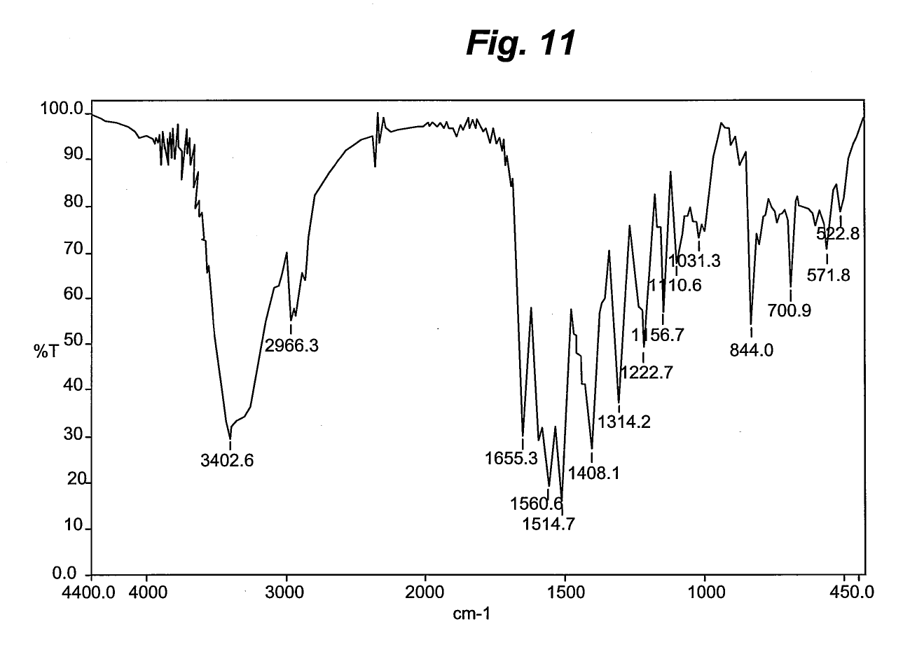

In another aspect, provided herein is a crystalline polymorph of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid hemi calcium salt, designated “Form III.” Form III may have the X-ray diffraction pattern of FIG. 3, the differential scanning calorimetry curve of FIG. 7, and the infrared spectrum of FIG. 11. The diffraction angles and relative intensities of the X-ray diffraction patterns of Form III are shown in Table 3 (in Example 4). For example, Form III can be characterized by an X-ray diffraction pattern having peaks at about characterized by an X-ray diffraction pattern having peaks at about 4.72, 7.01, 9.38, 13.59, 18.28, 19.56, 20.48, 22.33, 22.97, 23.51 and 27.29 degrees two theta or by an X-ray diffraction pattern having peaks at about 3.71, 4.72, 7.01, 7.35, 9.38, 10.16, 13.06, 13.59, 14.03, 14.57, 15.85, 17.09, 17.64, 18.28, 19.56, 20.48, 22.33, 22.97, 23.51, 27.29°2θ°. Form III can also be characterized by IR bands at 3402, 2966, 1655, 1560, 1514, 1222, 1156, 1110, 1031, 844 and 700 cm⁻¹. Further, Form III can be characterized by a differential scanning calorimetry curve that exhibits an endotherm with an extrapolated onset temperature of about 178.49° C. and associated heat of about 18.14 J/gram.

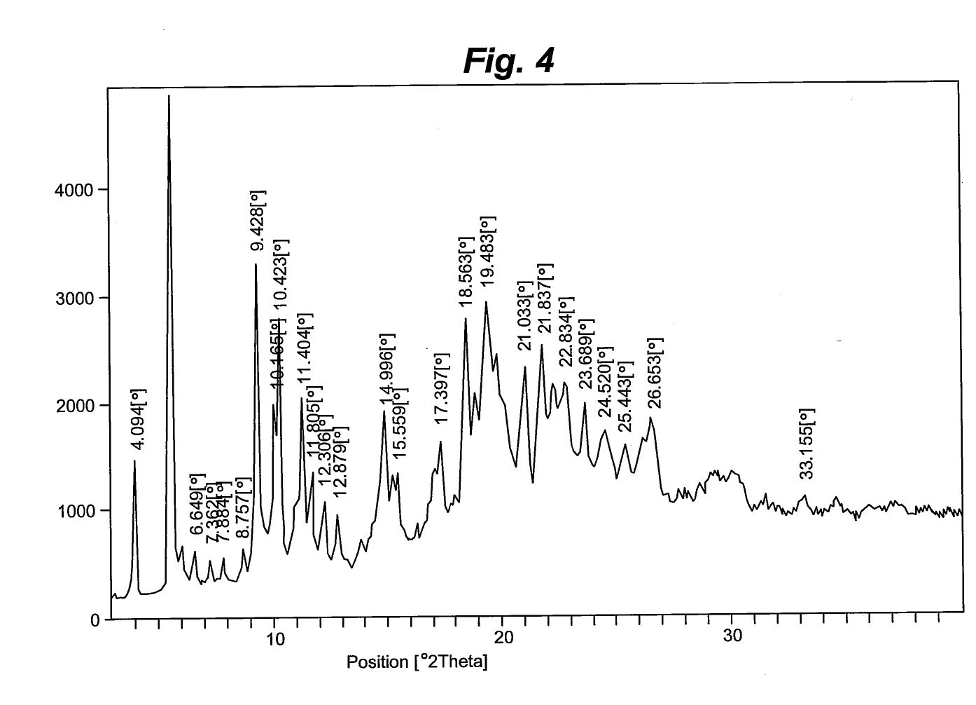

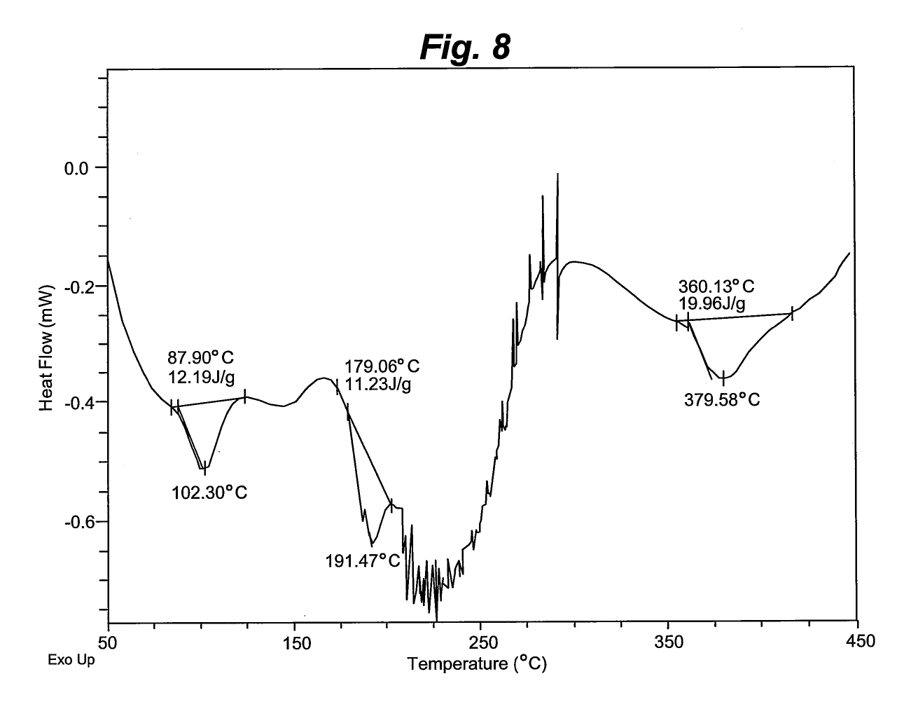

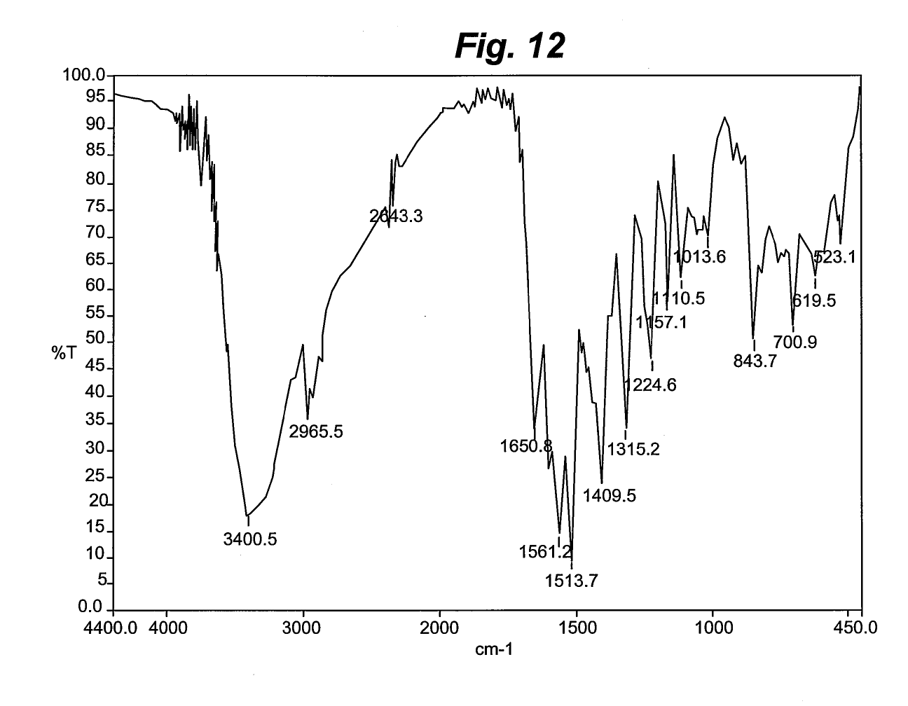

In another aspect, provided herein is a crystalline polymorph of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid hemi calcium salt, designated as “Form IV.” Form IV may have the X-ray diffraction pattern of FIG. 4, the differential scanning calorimetry curve of FIG. 8, and the infrared spectrum of FIG. 12. The diffraction angles and relative intensities of the X-ray diffraction patterns of Form IV are shown in Table 4 (in example 5). For example, Form IV can be characterized by an X-ray diffraction pattern having peaks at about 5.72, 9.42, 10.16, 10.42, 11.40, 18.56, 19.48, 21.03 and 21.83 degrees two theta or by an X-ray diffraction pattern having peaks at about 4.09, 5.72,9.42, 10.16, 10.42, 11.40, 11.80, 14.99, 17.39, 18.56, 19.48, 21.03, 21.83, 22.83° 2θ°. Form IV can also be characterized by IR bands at 3400, 2965, 2343, 1650, 1563, 1409, 1013 and 619 cm⁻¹. Further, Form IV can be characterized by a differential scanning calorimetry curve, which exhibits an endotherm with an extrapolated onset temperature of about 179° C. and associated heat of about 11.23 J/gram.

These X-ray diffraction patterns, infrared spectral bands and DSC data show that polymorphic Form I, Form II, Form III and Form IV, described herein, are different from each other.

Another aspect of the present invention provides processes for preparing the polymorphic forms of (3R,5)-7-[2-(4-fluorophenyl)-5-isopropyl-3-phenyl-4-[(4-hydroxymethylphenyl amino)carbonyl]-pyrrol-1-yl]-3,5-dihydroxy-heptanoic acid, hemi calcium salt, described herein.

The processes include (i) preparing a solution of amorphous forms, or any polymorphic form of (3R,5)-7-[2-(4-fluorophenyl)-5-isopropyl-3-phenyl-4-[(4-hydroxy methylphenylamino)carbonyl]-pyrrol-1-yl]-3,5-dihydroxy-heptanoic acid hemi calcium salt including solvates, anhydrous solutions, and solutions including one or more solvents, (ii) recovering the polymorphic forms described herein from the solution by the removal of the solvent(s), and (iii) optionally drying the polymorphic product so obtained.

The amorphous forms, and hydrates thereof, can be prepared following the processes described in PCT Publication Nos. WO 2004/106299, WO 2007/054790 and WO 2007/054896, incorporated herein by reference.

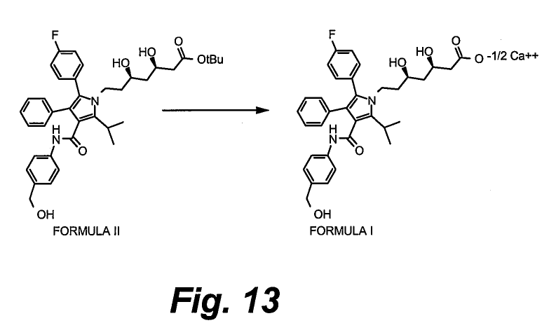

The crystalline polymorphic Form I of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, (Formula I) can be prepared via the scheme depicted in FIG. 13. Referring to FIG. 13, the compound of Formula II can be prepared following the procedures described in PCT Publication Nos. WO 2004/106299, WO 2007/054790 and WO 2007/054896. The compound of Formula II can be hydrolyzed with sodium hydroxide to form sodium salt in situ. The sodium salt, generated in situ, can be converted into its hemi calcium salt using, for example, calcium acetate, calcium hydroxide or calcium chloride.

The crystalline polymorphic Forms, for example Form I, can be obtained by dissolving a compound of Formula I in one or more solvents. Form I can be recovered from the solution by precipitation and filtration. The product may then be dried.

The solvent(s) used may be selected from one or more of acetates (e.g., ethyl acetate or isopropyl acetate), polar protic solvents (e.g., alcohols including methanol, ethanol, isopropanol or water) polar aprotic solvents (e.g., dimethylsulfoxide or dimethylformamide), esters (e.g., ethyl acetate or isopropyl acetate), ethers (e.g., diethyl ether, dioxane or tetrahydrofuran), ketones (e.g., acetone, 2-butanone or 4-methylpentanone), nitrites (e.g., acetonitrile or propionitrile), hydrocarbons (e.g., hexane or heptane), aromatic hydrocarbons (e.g., toluene or xylene), or mixtures thereof. The alcohol may include one or more of primary, secondary or tertiary alcohols having from one to six carbon atoms, for example, methanol, ethanol, denatured spirit, n-propanol, isopropanol, n-butanol, isobutanol, or t-butanol.

Additional solvent(s), in which the polymorphic forms of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, are insoluble or sparingly soluble, can optionally be added to the solution to precipitate the crystalline polymorphic forms before the removal of the solvent and recovering the polymorphic forms. The precipitation can be induced by reducing the temperature of the solvent, especially if the initial temperature is elevated. The precipitation may also be facilitated by adding seed crystals of forms described herein, by reducing the volume of the solution or by other means known in the art.

The amount of the solvent used is not limited and will vary depending on such conditions as the type of solvent, size of the batch and container, temperature of the reaction, and presence and absence of stirring. The crystallization temperature is not limited either, but good results can be obtained by conducting crystallization between 0° C. (the temperature of an ice-cold water bath) and room temperature (approximately 25° C.).

The product can be collected by any method in the art, for example, distillation, distillation under vacuum, evaporation, filtration, and filtration under vacuum, decantation, centrifugation or drying. The product obtained may be washed with a suitable solvent and it may be further or additionally dried to achieve desired moisture values. For example, the product may be further or additionally dried in a tray drier, dried under vacuum and/or in a fluid bed dryer. It may be dried under conditions that avoid degradation of the product, for example, air drying below 40° C., or at reduced pressure. Drying can also be carried out at elevated temperature or ambient temperature.

The processes may include one or more of the following embodiments. For example, crystalline polymorphic “Form I” can generally be prepared by charging or suspending in an organic solvent, such as an acetate (e.g., ethyl acetate or isopropyl acetate) or lower alcohol (e.g., methanol, ethanol or isopropanol) an amorphous form of the product obtained by the scheme shown in FIG. 13 and described above. Preferably, the organic solvent contains some water as a further solvent. The amount of water may range from about 40% to about 75%, preferably from about 50% to about 67%. It is also preferred that the suspension or solution may be heated at a temperature between about 50° C. and reflux temperature for a period of from about 1 hour to about 20 hours.

In another embodiment, crystalline polymorphic Form II can be prepared by suspending Form I, or amorphous forms, in an organic solvent, such as nitrites (e.g., acetonitrile or propionitrile). In this embodiment, the organic solvent preferably contains some water as a further solvent. The amount of water may range from about 40% to about 70%, and preferably from about 50% to about 60%. It is also preferred that the suspension be heated at temperature from about 50° C. to reflux temperature for a period of from about 1 hour to 20 hours.

In another embodiment, crystalline polymorphic Form III can be prepared by suspending Form I, or amorphous forms, in a polar protic solvent, like water. Preferably, the suspension is heated at temperatures from about 60° C. to reflux temperature for a period of from about 1 hour to about 10 hours.

In another embodiment, crystalline polymorphic Form IV can be prepared by suspending Form I, or amorphous forms, in an organic solvent, such as acetones (e.g., acetone, 2-butanone or 4-methylpentanone). It is preferred that the organic solvent contains some water as a further solvent. The amount of water may range from about 40% to about 75%, and preferably from about 50% to about 68%. Preferably, the suspension is heated at temperatures from about 40° C. to reflux temperature for a period of from about 1 hour to about 20 hours.

The polymorphic forms described herein are non-sticky and have excellent filtering properties, enabling easy scraping and handling of the filter cake. These forms have good flowability and are thus suitable for formulation into pharmaceutical dosage forms.

Another aspect of the present invention provides a pharmaceutical composition containing one or more polymorphic forms of the hemi calcium salt of (3R,5)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, optionally together with one or more pharmaceutically acceptable carriers, diluents, excipients or mixtures thereof.

The pharmaceutical compositions of the present invention, both those containing one polymorphic form and those containing two or more polymorphic forms, may be suitable for oral, buccal, rectal, inhalant, tropical, transdermal, ophthalmic, parenteral (e.g., subcutaneous, intramuscular or intravenous) administration or combination thereof. Although the most suitable route in any given case will depend upon the nature and severity of the condition being treated, the most preferred route of administration is oral.

The compositions may be formulated to provide immediate or sustained release of the therapeutic compounds. The compounds described herein can be administered alone but will generally be administered as an admixture with one or more pharmaceutically acceptable carriers, diluents, excipients or mixture thereof. The dosage forms include solid dosage forms or liquid dosage forms.

Solid dosage forms for oral administration may include capsules, tablets, pills, powder, granules or suppositories. For solid form preparations, the active compound is mixed with at least one inert, pharmaceutically acceptable excipient or carrier, for example, sodium citrate, dicalcium phosphate and/or a filler, an extender, for example, starch, lactose, sucrose, glucose, mannitol or silicic acid; binders, for example, carboxymethyl cellulose, alginates, gelatins, polyvinylpyrrolidone, sucrose, or acacia; disintegrating agents, for example, agar-agar, calcium carbonate, potato starch, aliginic acid, certain silicates or sodium carbonate; absorption accelerators, for example, quaternary ammonium compounds; wetting agents, for example, cetyl alcohol, glycerol, or mono stearate adsorbents, for example, Kaolin; lubricants, for example, talc, calcium stearate, magnesium stearate, solid polyethylene glycol, or sodium lauryl sulphate, and mixtures thereof. In embodiments in which the dosage formulations are prepared as capsules, tablets, or pills, the dosage form may also contain buffering agents.

The solid preparation of tablets, capsules, pills, or granules can be accomplished with coatings and/or shells, for example, film coatings, enteric coatings and other coatings well known in the pharmaceutical formulating art.

Liquid dosage forms for oral administration can include pharmaceutically acceptable emulsions, solutions, suspensions, syrups and elixirs. For liquid form preparations, the active compound can be mixed with water or other solvent, solubilizing agents and emulsifiers, for example, ethanol, isopropanol, ethyl carbonate, ethyl acetate, benzyl alcohol, benzyl benzoate, propylene glycol, 1,3-butylene glycol, dimethyl formamide, oils (for example, cottonseed, ground corn, germ, live, caster and sesame oil), glycerol and fatty acid ester of sorbitan and mixture thereof.

Besides inert diluents, the oral compositions can also include adjuvants, for example, wetting agents, emulsifying agents, suspending agents, sweetening agents, flavoring agents and perfuming agents.

Injectable preparations, for example, sterile injections, aqueous suspensions may be formulated according to the art using suitable dispersing or wetting and suspending agents. Among the acceptable vehicles and solvents that may be employed are water, Ringer's solution and isotonic sodium chloride.

The dosage forms for buccal, rectal, inhalant, tropical, transdermal, ophthalmic and parenteral administration can be prepared following the procedures known in the formulary art.

The formulations as described herein may be formulated so as to provide quick, sustained, or delayed release of the active compound after administration to the patient by employing procedures well-known to the art. The term “patient” as used herein refers to a human or non-human mammal, which is the object of treatment, observation or experiment.

The pharmaceutical preparations can be in unit dosage forms, and in such forms, the preparations are subdivided into unit doses containing appropriate quantities of an active compound.

The amount of a compound described herein that will be effective in the treatment of a particular disorder or condition can be determined by standard clinical techniques. In addition, in vitro or in vivo assays may optionally be employed to help identify optimal dosage ranges.

Another aspect of the present invention provides a method for treating a patient suffering from cholesterol-related disease(s), diabetes and related disease(s), cerebrovascular disease(s) or cardiovascular disease(s), that includes administering to a patient a therapeutically effective amounts of one or more compounds or pharmaceutical compositions described herein.

The compounds or pharmaceutical compositions described herein can be used for treating diseases or disorders, for example, arteriosclerosis, atherosclerosis, hypercholesterolemia, hyperlipidemia, hyperlipoproteinemia, hypertriglyceridemia, hypertension, stroke, ischemia, endothelium dysfunction, peripheral vascular disease, peripheral arterial disease, coronary heart disease, myocardial infarction, cerebral infarction, myocardial microvascular disease, dementia, Alzheimer's disease, osteoporosis, osteopenia, angina or restenosis.

Examples set forth below demonstrate general synthetic procedures for preparation of polymorphic forms. In each instance, X-ray diffraction data were collected as follows:

XRD: Instrument: Model RU-H3R (Riigaku),

Data collection parameters: Voltage: 50KV; current: 120 mA; scan speed: 2°/min; scan step: 0.02°; scan range: 3-40°. XRD data are shown in tables 1-4.

IR: Instrument: FTIR Paragon 1000PC.

Data collection parameters: Medium: KBr; Scanning range: 440-4400 cm⁻¹.

DSC: Instrument: Thermal Analyser Q 100

Data collection parameters: Scanning rate: 10° C./min; Temperature: 50° C.-300° C.

The examples are provided to illustrate particular aspects of the disclosure, and do not constrain the scope of the present invention.

### EXAMPLES

### Example 1

### Preparation of Crystalline Polymorphic Form I

Referring to FIG. 13, a compound of Formula II was hydrolyzed using sodium hydroxide to form the sodium salt in situ, which was in the aqueous layer. This aqueous layer was extracted with ethyl acetate to remove any impurities. The aqueous layer containing the sodium salt was reacted with calcium acetate at room temperature under stirring to form the precipitate of compound of Formula I. To the reaction vessel an equal amount of ethyl acetate was charged and the reaction mixture was heated to reflux under stirring to dissolve all the precipitated compound of Formula I. The hot solution was filtered and allowed to cool to about 25° C. to about 30° C. under stirring and continued to stir for about 4 to 5 hours. The product was then filtered, washed with ethyl acetate and deionized water and unloaded for drying. The product was dried for about 10 hours to about 12 hours at about 60° C. in a vacuum tray dryer to give the desired crystalline polymorphic Form I.

### Example 2

### Preparation of Crystalline Polymorphic Form I

The well suspended amorphous form of the compound of Formula I (75 gm) in ethanol (375 mL, 5 times) was heated at about 50° C. to about 55° C. until a clear solution was obtained. Deionized water (375 mL, 5 times) was added to cool the solution to room temperature, and the solution was heated to about 50° C. to about 55° C. for about 1 hour. The milky white solution was then allowed to cool to between about 25° C. to about 30° C. and stirred for about two and half hours. Further, deionized water (375 ml, 5 times) was slowly added and stirred for about half an hour. The solid was filtered, washed with deionized water and hexane, and dried under vacuum at about 55° C. to about 60° C. for about 10 to about 12 hours to form crystalline polymorphic Form I. Diffraction angles and relative intensities for the X ray diffraction patterns of Form I are shown in Table 1.

TABLE 1
XRD diffraction pattern of Form I (Ethyl acetate:Water, 1:1)

| S. No. | Diffraction angle (2θ°) | Intensity (I/Iₒ) |
|---|---|---|
| 1 | 3.99 | 15.57 |
| 2 | 5.43 | 38.06 |
| 3 | 5.74 | 17.16 |
| 4 | 7.95 | 39.42 |
| 5 | 9.61 | 100 |
| 6 | 11.29 | 50.51 |
| 7 | 11.92 | 60.87 |
| 8 | 15.91 | 24.68 |
| 9 | 18.91 | 37.80 |
| 10 | 19.25 | 44.65 |
| 11 | 22.78 | 44.39 |
| 12 | 23.95 | 38.86 |
| 13 | 28.02 | 27.20 |

### Example 3

### Preparation of Crystalline Polymorphic Form II

The amorphous form (3.0 gm) was dissolved in fifty percent acetonitrile in water (36 mL, 12 times) at refluxing temperature under stirring. The solution was again stirred for about 0.5 hour at reflux temperature. The hot solution was cooled to between about 25° C. to about 30° C. and stirred for 8 to 10 hours, filtered, washed with deionized water, and dried under vacuum for about 10 to about 12 hours at about 55° C. to about 60° C. to form crystalline polymorphic Form II. Diffraction angles and relative intensities for the X ray diffraction patterns of Form II are shown in Table 2.

TABLE 2
XRD diffraction pattern of Form II (acetonitrile:water, 1:1)

| S. No. | Diffraction angle (2θ°) | Intensity (I/Iₒ) |
|---|---|---|
| 1 | 3.76 | 63.85 |
| 2 | 5.32 | 14.84 |
| 3 | 6.08 | 43.71 |
| 4 | 7.19 | 46.52 |
| 5 | 8.90 | 65.23 |
| 6 | 9.34 | 32.36 |
| 7 | 11.27 | 26.66 |
| 8 | 12.30 | 32.96 |
| 9 | 12.86 | 46.52 |
| 10 | 15.29 | 18.51 |
| 11 | 16.18 | 17.79 |
| 12 | 17.62 | 30.60 |
| 13 | 20.16 | 100 |
| 14 | 21.08 | 26.47 |
| 15 | 21.51 | 26.64 |
| 16 | 22.57 | 24.55 |
| 17 | 24.41 | 77.94 |
| 18 | 24.63 | 29.26 |
| 19 | 25.15 | 23.13 |
| 20 | 26.59 | 35.24 |
| 21 | 28.77 | 27.98 |
| 22 | 35.67 | 11.77 |
| 23 | 37.48 | 14.78 |

### Example 4

### Preparation of Crystalline Polymorphic Form III

The suspended amorphous form (10 gm) in water (200 mL, 20 times) was subjected to reflux under stirring for about 2 hours. The suspension was cooled to between about 25° C. to about 30° C. and stirred for about 2 to about 3 hours, filtered, and washed with deionized water to form crystalline polymorphic Form III. The crystalline form was finally dried at about 55° C. to about 60° C. under vacuum for about 10 to 12 hours. Diffraction angles and relative intensities for the X ray diffraction patterns of Form III are shown in Table 3.

TABLE 3
XRD diffraction pattern of Form III from Amorphous form (Water)

| S. No. | Diffraction angle (2θ°) | Intensity (I/Iₒ) |
|---|---|---|
| 1 | 3.71 | 18.87 |
| 2 | 4.72 | 29.25 |
| 3 | 7.01 | 18.91 |
| 4 | 7.35 | 10.07 |
| 5 | 9.38 | 100 |
| 6 | 10.16 | 16.65 |
| 7 | 13.06 | 9.92 |
| 8 | 13.59 | 13.17 |
| 9 | 14.03 | 13.80 |
| 10 | 14.57 | 9.09 |
| 11 | 15.85 | 16.40 |
| 12 | 17.09 | 9.46 |
| 13 | 17.64 | 10.95 |
| 14 | 18.28 | 33.40 |
| 15 | 19.56 | 23.73 |
| 16 | 20.48 | 47.94 |
| 17 | 22.33 | 29.09 |
| 18 | 22.97 | 21.97 |
| 19 | 23.51 | 18.39 |
| 20 | 27.29 | 19.22 |

### Example 5

### Preparation of Crystalline Polymorphic Form IV

Deionized water (50 mL, 10 times) was charged slowly to a well-stirred suspension of the amorphous form of the compound of Formula I (5 gm) in acetone (25 mL, 5 times) at refluxing temperature. The clear solution was refluxed for about 30 minutes and then allowed to cool to between about 25° C. to about 30° C. under stirring. The solution was stirred at room temperature for about 3 days, filtered the white solid, washed with deionized water, and dried under vacuum at about 55° C. to about 60° C. for about 8 to about 10 hours to form crystalline polymorphic Form IV. Diffraction angles and relative intensities for the X ray diffraction patterns of Form IV are shown in Table 4.

TABLE 4
XRD diffraction pattern of Form IV (Acetone:water, 1:2)

| S. No. | Diffraction angle (2θ°) | Intensity (I/Iₒ) |
|---|---|---|
| 1 | 4.09 | 27.17 |
| 2 | 5.72 | 100 |
| 3 | 9.42 | 65.21 |
| 4 | 10.16 | 34.89 |
| 5 | 10.42 | 51.66 |
| 6 | 11.40 | 35.23 |
| 7 | 11.80 | 19.54 |
| 8 | 14.99 | 27.85 |
| 9 | 17.39 | 20.94 |
| 10 | 18.56 | 45.55 |
| 11 | 19.48 | 48.65 |
| 12 | 21.03 | 33.64 |
| 13 | 21.83 | 36.73 |
| 14 | 22.83 | 28.27 |

### Example 6

### Preparation of Amorphous Form from Crystalline Polymorphic Form I

The clear solution of Form I (30 gm) in methanol (150 mL, 5 times) was stirred at room temperature for about one hour. The methanol solution was concentrated to dryness to give the amorphous form. The amorphous form thus obtained was dried under vacuum at about 60° C. for about 24 hours.

### Example 7

### Preparation of Crystalline Polymorphic Form I from Amorphous Form

The amorphous form (900 gm) in ethyl acetate:water (9 Lt, 1:1, 10 times) was refluxed for about 2 hours. The hot solution was cooled to 45° C. under stirring and again stirred at room temperature for about 2 to about 3 hours, filtered, washed with deionized water, and dried at about 55° C. to about 60° C. for about 8 to 10 hours.

### Example 8

### Stability Testing of Amorphous and Polymorphic Form

The integrity of the different forms of the (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, was tested under different atmospheric conditions to determine the stability of the amorphous and polymorphic form of the drug in various storage environments. Reversed Phase-HPLC (RP-HPLC) was used to separate (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, from smaller molecules representing breakdown products as well as oxidized drug. The relative amount of the drug was reported as a percent of total absorption by UV. The total peak area of all UV absorption impurities was used to define total impurity of the drug. Impurities are defined by their relative retention time (RRT) compared to native drug. Samples were injected onto a C18 column using standard temperature, gradient and run-time conditions.

The results of this integrity testing for the amorphous form of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, is shown in Table 5.

Three separate batches of polymorphic Form I of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, were prepared and tested under the same atmospheric conditions described for the amorphous form. The results of the integrity testing of those three batches of Form I are shown in Tables 6-8.

TABLE 5
Integrity Testing of Amorphous form

| Batch #/ | RRT | RRT | RRT | RRT | RRT | RRT | RRT | RRT | RRT | RRT | RRT | RRT | RRT | RRT | RRT | RRT | RRT | RRT | |
|---|---|---|---|---|---|---|---|---|---|---|---|---|---|---|---|---|---|---|---|
| Condition | 0.731 | 0.738 | 0.832 | 0.876 | 0.885 | 0.892 | 0.949 | 0.952 | 0.965 | 0.974 | 0.981 | 0.991 | 1.024 | 1.041 | 1.047 | 1.050 | 1.074 | 1.104 | Total |
| Initial | — | — | — | 0.146 | — | — | — | — | 0.560 | — | — | 0.022 | 0.464 | 0.581 | — | — | — | — | 1.703 |
| PP00204 - | 0.030 | — | 0.034 | 0.150 | — | — | — | 0.040 | 0.532 | 0.052 | — | 0.022 | 0.459 | 0.585 | — | — | — | — | 1.904 |
| 5° C.- | | | | | | | | | | | | | | | | | | | |
| 1 month | | | | | | | | | | | | | | | | | | | |
| PP00204 - | 0.038 | — | 0.037 | 0.145 | — | 0.038 | — | 0.042 | 0.512 | 0.054 | — | 0.018 | 0.464 | 0.587 | — | — | 0.021 | — | 1.956 |
| 30° C./ | | | | | | | | | | | | | | | | | | | |
| 65% RH- | | | | | | | | | | | | | | | | | | | |
| 1 month | | | | | | | | | | | | | | | | | | | |
| PP00204 - | 0.039 | — | 0.034 | 0.125 | — | 0.055 | — | — | 0.526 | 0.071 | — | 0.024 | 0.464 | 0.565 | — | — | 0.032 | — | 1.955 |
| 40° C./ | | | | | | | | | | | | | | | | | | | |
| 75% RH- | | | | | | | | | | | | | | | | | | | |
| 1 month | | | | | | | | | | | | | | | | | | | |
| PP00204 - | — | — | — | — | 0.177 | — | — | 0.048 | 0.501 | 0.067 | — | 0.029 | 0.508 | 0.604 | — | — | — | — | 1.934 |
| 5° C.- | | | | | | | | | | | | | | | | | | | |
| 2 months | | | | | | | | | | | | | | | | | | | |
| PP00204 - | — | — | — | — | 0.154 | 0.119 | — | 0.050 | 0.505 | 0.042 | — | 0.022 | 0.524 | 0.611 | — | — | — | — | 2.027 |
| 30° C./ | | | | | | | | | | | | | | | | | | | |
| 65% RH- | | | | | | | | | | | | | | | | | | | |
| 2 months | | | | | | | | | | | | | | | | | | | |
| PP00204 - | — | 0.042 | — | — | 0.159 | 0.127 | 0.063 | — | 0.547 | — | — | 0.027 | 0.543 | 0.622 | — | — | 0.077 | — | 2.207 |
| 40° C./ | | | | | | | | | | | | | | | | | | | |
| 75% RH- | | | | | | | | | | | | | | | | | | | |
| 2 months | | | | | | | | | | | | | | | | | | | |
| PP00204 - | — | — | — | — | — | 0.161 | — | — | 0.550 | — | 0.058 | — | 0.506 | 0.635 | — | — | — | — | 1.91 |
| 5° C. - | | | | | | | | | | | | | | | | | | | |
| 3 months | | | | | | | | | | | | | | | | | | | |
| PP00204 - | — | — | — | — | 0.145 | 0.212 | — | — | 0.542 | — | 0.058 | — | 0.518 | 0.648 | — | — | — | — | 2.123 |
| 25° C./ | | | | | | | | | | | | | | | | | | | |
| 60% RH - | | | | | | | | | | | | | | | | | | | |
| 3 months | | | | | | | | | | | | | | | | | | | |
| PP00204 - | — | — | 0.046 | — | — | 0.184 | — | — | 0.542 | — | 0.057 | — | 0.516 | 0.638 | — | — | — | — | 1.983 |
| 30° C./ | | | | | | | | | | | | | | | | | | | |
| 65% RH- | | | | | | | | | | | | | | | | | | | |
| 3 months | | | | | | | | | | | | | | | | | | | |
| PP00204 - | — | — | — | — | — | 0.189 | — | — | 0.548 | — | 0.065 | — | 0.515 | 0.632 | — | — | — | 0.074 | 2.023 |
| 40° C./ | | | | | | | | | | | | | | | | | | | |
| 75% RH- | | | | | | | | | | | | | | | | | | | |
| 3 months | | | | | | | | | | | | | | | | | | | |
| PP00204 - | 0.037 | — | 0.028 | 0.014 | — | 0.016 | — | 0.024 | 0.552 | — | — | — | 0.582 | 0.592 | — | — | 0.028 | — | 1.873 |
| 5° C. - | | | | | | | | | | | | | | | | | | | |
| 6 months | | | | | | | | | | | | | | | | | | | |
| PP00204 - | 0.031 | — | 0.029 | 0.133 | — | 0.124 | — | 0.037 | 0.546 | — | — | — | 0.618 | 0.593 | — | — | 0.068 | 0.037 | 2.216 |
| 25° C./ | | | | | | | | | | | | | | | | | | | |
| 60% RH- | | | | | | | | | | | | | | | | | | | |
| 6 months | | | | | | | | | | | | | | | | | | | |
| PP00204 - | — | — | — | — | 0.187 | 0.168 | — | — | 0.699 | — | — | — | 0.693 | 0.777 | — | — | 0.103 | 0.032 | 2.659 |
| 30° C./ | | | | | | | | | | | | | | | | | | | |
| 55% RH- | | | | | | | | | | | | | | | | | | | |
| 6 months | | | | | | | | | | | | | | | | | | | |
| PP00204 - | 0.031 | — | 0.031 | 0.152 | — | 0.19 | — | 0.022 | 0.497 | — | — | — | 0.460 | 0.573 | — | 0.094 | 0.025 | 0.037 | 2.112 |
| 40° C./ | | | | | | | | | | | | | | | | | | | |
| 75% RH- | | | | | | | | | | | | | | | | | | | |
| 6 months | | | | | | | | | | | | | | | | | | | |

TABLE 6
Integrity Testing of Batch 1 of Polymorphic Form I

| | | | | | | | | | | | | | | High- | |
|---|---|---|---|---|---|---|---|---|---|---|---|---|---|---|---|
| | | | Assay | Water | | | | | | | | | | est | Total |
| | | Descrip- | (% | (% | RRT | RRT | RRT | RRT | RRT | RRT | RRT | RRT | RRT | Impu- | Impu- |
| Batch # | Condition | tion | W/W) | W/W) | 0.720 | 0.935 | 1.045 | 1.094 | 1.180 | 1.437 | 1.593 | 1.64 | 1.694 | rity | rity |
| | Initial | White | 98.48 | 4.55 | 0.046 | .0411 | 0.385 | 0.142 | — | 0.162 | 0.079 | 0.046 | 0.068 | 0.411 | 1.340 |
| | | Powder | | | | | | | | | | | | | |
| PP00405 | 5° C. 1M | White | 98.31 | 4.81 | — | 0.360 | 0.357 | 0.157 | — | 0.155 | 0.082 | 0.057 | 0.069 | 0.360 | 1.237 |
| | | Powder | | | | | | | | | | | | | |
| PP00405 | 30° C./65% | White | 98.25 | 4.16 | — | 0.370 | 0.300 | 0.149 | — | 0.161 | 0.061 | 0.047 | 0.060 | 0.370 | 1.228 |
| | RH 1M | Powder | | | | | | | | | | | | | |
| PP00405 | 40° C./75% | White | 98.22 | 4.52 | — | 0.405 | 0.39 | 0.167 | — | 0.155 | 0.085 | 0.05 | 0.071 | 0.405 | 1.323 |
| | RH 1M | Powder | | | | | | | | | | | | | |
| PP00405 | 5° C./2M | White | 98.25 | 4.61 | — | 0.380 | 0.375 | 0.161 | 0.048 | 0.164 | 0.085 | 0.051 | 0.067 | 0.380 | 1.33 |
| | | Powder | | | | | | | | | | | | | |
| PP00405 | 30° C./65% | White | 98.23 | 3.85 | — | 0.390 | 0.374 | 0.16 | 0.044 | 0.163 | 0.080 | 0.044 | 0.062 | 0.390 | 1.320 |
| | RH 2M | Powder | | | | | | | | | | | | | |
| PP00405 | 40° C./75% | White | 98.16 | 4.63 | — | 0.407 | 0.408 | 0.164 | 0.049 | 0.169 | 0.082 | 0.046 | 0.067 | 0.408 | 1.39 |
| | RH 2M | Powder | | | | | | | | | | | | | |
| PP00405 | 5° C. 3M | White | 98.20 | 4.97 | — | 0.385 | 0.469 | 0.149 | — | 0.169 | 0.077 | 0.047 | 0.056 | 0.469 | 1.35 |
| | | Powder | | | | | | | | | | | | | |
| PP00405 | 25° C./60% | White | 98.21 | 4.03 | — | 0.368 | 0.382 | 0.153 | — | 0.155 | 0.079 | 0.047 | 0.064 | 0.388 | 1.270 |
| | RH 3M | Powder | | | | | | | | | | | | | |
| PP00405 | 30° C./65% | White | 98.15 | 4.73 | — | 0.416 | 0.405 | 0.167 | — | 0.168 | 0.081 | 0.048 | 0.066 | 0.416 | 1.350 |
| | RH 3M | Powder | | | | | | | | | | | | | |
| PP00405 | 40° C./75% | White | 98.11 | 4.64 | — | 0.403 | 0.396 | 0.162 | — | 0.167 | 0.094 | 0.049 | 0.056 | 0.403 | 1.330 |
| | RH 3M | Powder | | | | | | | | | | | | | |

| | | | | | | | | | | | | | | | High- | |
|---|---|---|---|---|---|---|---|---|---|---|---|---|---|---|---|---|
| | | | Assay | Water | | | | | | | | | | | est | Total |
| | | Descrip- | (% | (% | RRT | RRT | RRT | RRT | RRT | RRT | RRT | RRT | RRT | RRT | Impu- | Impu- |
| Batch # | Conditions | tion | W/W) | W/W) | 0.136 | 0.935 | 1.045 | 1.094 | 1.180 | 1.437 | 1.550 | 1.593 | 1.640 | 1.694 | rity | rity |
| PP00405 | 5° C./4M | White | 98.24 | 4.43 | — | 0.419 | 0.409 | 0.169 | — | 0.132 | — | 0.071 | 0.067 | 0.071 | 0.419 | 1.338 |
| | | Powder | | | | | | | | | | | | | | |
| PP00405 | 5° C./5M | White | 98.29 | 5.55 | — | 0.289 | 0.312 | 0.105 | — | 0.155 | 0.587 | — | 0.024 | 0.045 | 0.587 | 1.517 |
| | | Powder | | | | | | | | | | | | | | |
| PP00405 | 5° C./6M | White | 98.02 | 5.04 | — | 0.423 | 0.390 | 0.153 | — | 0.170 | — | 0.081 | 0.053 | 0.070 | 0.423 | 1.340 |
| | | Powder | | | | | | | | | | | | | | |
| PP00405 | 25° C./50% | White | 98.01 | 5.41 | — | 0.390 | 0.370 | 0.140 | — | 0.117 | — | 0.073 | 0.048 | 0.074 | 0.390 | 1.212 |
| | RH 6M | Powder | | | | | | | | | | | | | | |
| PP00405 | 30° C./65% | White | 98.08 | 5.62 | 0.049 | 0.355 | 0.361 | 0.140 | — | 0.150 | — | 0.077 | 0.042 | 0.044 | 0.361 | 1.218 |
| | RH 6M | Powder | | | | | | | | | | | | | | |
| PP00405 | 40° C./75% | White | 98.22 | 5.55 | — | 0.391 | 0.351 | 0.146 | — | 0.151 | — | 0.098 | 0.031 | 0.074 | 0.391 | 1.242 |
| | RH 6M | Powder | | | | | | | | | | | | | | |
| PP00405 | 5° C. 9M | White | 98.12 | 5.07 | — | 0.407 | 0.407 | 0.161 | 0.055 | 0.170 | — | 0.065 | 0.033 | 0.064 | 0.407 | 1.362 |
| | | Powder | | | | | | | | | | | | | | |
| PP00405 | 25° C./60% | White | 98.09 | 5.04 | — | 0.374 | 0.376 | 0.141 | — | 0.162 | — | 0.076 | 0.046 | 0.068 | 0.376 | 1.243 |
| | RH 9M | Powder | | | | | | | | | | | | | | |
| PP00405 | 30° C./65% | White | 98.15 | 5.55 | 0.056 | 0.387 | 0.351 | 0.134 | — | 0.147 | — | 0.080 | 0.033 | 0.051 | 0.387 | 1.239 |
| | RH 9M | Powder | | | | | | | | | | | | | | |
| PP00405 | 5° C. 12M | White | 97.78 | 5.08 | — | 0.417 | 0.432 | 0.158 | 0.022 | 0.152 | — | 0.086 | 0.051 | 0.068 | 0.432 | 1.413 |
| | | Powder | | | | | | | | | | | | | | |
| PP00405 | 25° C./60% | White | 97.95 | 5.47 | — | 0.412 | 0.396 | 0.153 | 0.019 | 0.145 | — | 0.083 | 0.051 | 0.059 | 0.412 | 1.318 |
| | RH 12M | Powder | | | | | | | | | | | | | | |
| PP00405 | 30° C./65% | Descrip- | 98.09 | 5.91 | 0.240 | 0.375 | 0.372 | 0.120 | — | 0.148 | — | 0.075 | 0.044 | 0.055 | 0.375 | 1.492 |
| | RH 12M | tion | | | | | | | | | | | | | | |

TABLE 7
Integrity Testing of Batch 2 of Polymorphic Form I

| | | | | | | | | | | | | | High- | |
|---|---|---|---|---|---|---|---|---|---|---|---|---|---|---|
| | | | Assay | Water | | | | | | | | | est | Total |
| | | Descrip- | (% | (% | RRT | RRT | RRT | RRT | RRT | RRT | RRT | RRT | Impu- | Impu- |
| Batch # | Condition | tion | W/W) | W/W | 0.935 | 1.045 | 1.096 | 1.183 | 1.439 | 1.596 | 1.64 | 1.698 | rity | rity |
| | Initial | White | 98.45 | 4.92 | 0.413 | 0.457 | 0.229 | — | 0.168 | 0.075 | 0.057 | 0.062 | 0.457 | 1.46 |
| | | Powder | | | | | | | | | | | | |
| PP00105 | 5° C. 1M | White | 98.36 | 4.45 | 0.412 | 0.459 | 0.248 | 0.051 | 0.17 | 0.086 | 0.063 | 0.068 | 0.459 | 1.56 |
| | | Powder | | | | | | | | | | | | |
| PP00105 | 30° C./65% | White | 98.23 | 4.16 | 0.396 | 0.444 | 0.24 | — | 0.151 | 0.076 | 0.06 | 0.065 | 0.444 | 1.44 |
| | RH 1M | Powder | | | | | | | | | | | | |
| PP00105 | 40° C./75% | White | 98.15 | 4.71 | 0.404 | 0.444 | 0.244 | — | 0.153 | 0.080 | 0.061 | 0.062 | 0.444 | 1.45 |
| | RH 1M | Powder | | | | | | | | | | | | |
| PP00105 | 5° C. 2M | White | 98.24 | 4.31 | 0.413 | 0.443 | 0.233 | 0.06 | 0.163 | 0.079 | 0.06 | 0.07 | 0.443 | 1.52 |
| | | Powder | | | | | | | | | | | | |
| PP00105 | 30°/65% | White | 98.20 | 4.16 | 0.411 | 0.449 | 0.242 | 0.063 | 0.165 | 0.074 | 0.063 | 0.069 | 0.449 | 1.54 |
| | RH 2M | Powder | | | | | | | | | | | | |
| PP00105 | 40° C./75% | White | 98.19 | 4.35 | 0.416 | 0.457 | 0.234 | 0.072 | 0.175 | 0.079 | 0.066 | 0.065 | 0.457 | 1.56 |
| | RH 2M | Powder | | | | | | | | | | | | |
| PP00105 | 5° C. 3M | White | 98.19 | 3.90 | 0.376 | 0.414 | 0.216 | — | 0.150 | 0.068 | 0.055 | 0.068 | 0.414 | 1.35 |
| | | Powder | | | | | | | | | | | | |
| PP00105 | 25° C./60% | White | 98.15 | 4.74 | 0.383 | 0.427 | 0.214 | — | 0.158 | 0.067 | 0.051 | 0.065 | 0.427 | 1.37 |
| | RH 3M | Powder | | | | | | | | | | | | |
| PP00105 | 30° C./65% | White | 98.14 | 5.02 | 0.399 | 0.437 | 0.229 | — | 0.158 | 0.074 | 0.058 | 0.062 | 0.437 | 1.42 |
| | RH 3M | Powder | | | | | | | | | | | | |
| PP00105 | 40° C./75% | White | 98.09 | 4.45 | 0.413 | 0.442 | 0.229 | — | 0.165 | 0.078 | 0.056 | 0.06 | 0.442 | 1.44 |
| | RH 3M | Powder | | | | | | | | | | | | |

| | | | | | | | | | | | | | | High- | |
|---|---|---|---|---|---|---|---|---|---|---|---|---|---|---|---|
| | | | Assay | Water | | | | | | | | | | est | Total |
| | | Descrip- | (% | (% | RRT | RRT | RRT | RRT | RRT | RRT | RRT | RRT | RRT | Impu- | Impu- |
| Batch # | Condition | tion | W/W) | W/W | 0.138 | 0.935 | 1.045 | 1.096 | 1.183 | 1.439 | 1.596 | 1.640 | 1.698 | rity | rity |
| PP00105 | 5° C. 4M | White | 98.17 | 5.08 | 0.228 | 0.341 | 0.427 | 0.205 | — | 0.159 | 0.060 | 0.051 | 0.052 | 0.427 | 1.523 |
| | | Powder | | | | | | | | | | | | | |
| PP00105 | 5° C. 5M | White | 98.27 | 4.67 | 0.22 | 0.373 | 0.401 | 0.236 | — | 0.122 | 0.054 | 0.047 | 0.065 | 0.401 | 1.527 |
| | | Powder | | | | | | | | | | | | | |
| PP00105 | 5° C. 6M | White | 98.61 | 5.04 | 0.238 | 0.373 | 0.438 | 0.189 | 0.056 | 0.146 | 0.080 | 0.048 | 0.059 | 0.438 | 1.627 |
| | | Powder | | | | | | | | | | | | | |
| PP00105 | 25° C./60% | White | 98.15 | 5.04 | 0.113 | 0.338 | 0.405 | 0.233 | — | 0.142 | 0.066 | 0.035 | 0.056 | 0.405 | 1.388 |
| | RH 6M | Powder | | | | | | | | | | | | | |
| PP00105 | 30° C./65% | White | 98.09 | 4.96 | — | 0.409 | 0.460 | 0.218 | — | 0.156 | 0.070 | 0.054 | 0.063 | 0.460 | 1.430 |
| | RH 6M | Powder | | | | | | | | | | | | | |
| PP00105 | 40° C./75% | White | 98.14 | 5.45 | 0.270 | 0.377 | 0.410 | 0.217 | — | 0.146 | 0.073 | 0.045 | 0.052 | 0.410 | 1.590 |
| | RH 6M | Powder | | | | | | | | | | | | | |
| PP00105 | 5° C. 9M | White | 97.96 | 4.64 | 0.228 | 0.366 | 0.400 | 0.205 | — | 0.188 | 0.061 | 0.056 | 0.082 | 0.400 | 1.586 |
| | | Powder | | | | | | | | | | | | | |
| PP00105 | 25° C./60% | White | 98.06 | 5.31 | 0.111 | 0.375 | 0.389 | 0.212 | 0.039 | 0.155 | 0.042 | 0.045 | 0.063 | 0.389 | 1.431 |
| | RH 9M | Powder | | | | | | | | | | | | | |
| PP00105 | 30° C./65% | White | 98.07 | 5.10 | — | 0.404 | 0.442 | 0.232 | 0.036 | 0.182 | 0.053 | 0.058 | 0.048 | 0.442 | 1.455 |
| | RH 9M | Powder | | | | | | | | | | | | | |
| PP00105 | 5° C. 12M | White | 98.27 | 5.03 | 0.259 | 0.392 | 0.412 | 0.208 | 0.033 | 0.141 | 0.053 | 0.050 | 0.045 | 0.412 | 1.593 |
| | | Powder | | | | | | | | | | | | | |
| PP00105 | 25° C./60% | White | 98.25 | 5.02 | 0.199 | 0.365 | 0.407 | 0.212 | 0.036 | 0.152 | 0.046 | 0.052 | 0.053 | 0.407 | 1.522 |
| | RH 12M | Powder | | | | | | | | | | | | | |
| PP00105 | 30° C./65% | White | 98.35 | 4.87 | 0.200 | 0.399 | 0.360 | 0.192 | 0.048 | 0.163 | 0.065 | 0.048 | 0.045 | 0.399 | 1.520 |
| | RH 12M | Powder | | | | | | | | | | | | | |

TABLE 8
Integrity Testing of Batch 3 of Polymorphic Form I

| | | | | | | | | | | | | | | High- | |
|---|---|---|---|---|---|---|---|---|---|---|---|---|---|---|---|
| | | | Assay | Water | | | | | | | | | | est | Total |
| | | Descrip- | (% | (% | RRT | RRT | RRT | RRT | RRT | RRT | RRT | RRT | RRT | Impu- | Impu- |
| Batch # | Condition | tion | W/W) | W/W | 0.706 | 0.935 | 1.045 | 1.095 | 1.18 | 1.44 | 1.590 | 1.640 | 1.698 | rity | rity |
| | Initial | White | 98.43 | 4.76 | 0.045 | 0.428 | 0.349 | 0.159 | — | 0.235 | 0.093 | 0.054 | 0.084 | 0.428 | 1.438 |
| | | Powder | | | | | | | | | | | | | |
| PP00205 | 5° C./1M | White | 98.26 | 4.77 | 0.042 | 0.421 | 0.352 | 0.165 | — | 0.21 | 0.100 | 0.074 | 0.071 | 0.421 | 1.455 |
| | | Powder | | | | | | | | | | | | | |
| PP00205 | 30° C./65% | White | 98.21 | 4.47 | — | 0.449 | 0.385 | 0.162 | — | 0.234 | 0.102 | 0.071 | 0.072 | 0.449 | 1.495 |
| | RH 1M | Powder | | | | | | | | | | | | | |
| PP00205 | 40° C./75% | White | 98.23 | 4.33 | — | 0.452 | 0.372 | 0.162 | — | 0.215 | 0.100 | 0.071 | 0.066 | 0.452 | 1.46 |
| | RH 1M | Powder | | | | | | | | | | | | | |
| PP00205 | 5° C. 2M | White | 98.21 | 4.02 | — | 0.402 | 0.340 | 0.155 | — | 0.203 | 0.096 | 0.064 | 0.067 | 0.402 | 1.33 |
| | | Powder | | | | | | | | | | | | | |
| PP00205 | 30° C./65% | White | 98.27 | 4.59 | — | 0.416 | 0.362 | 0.163 | 0.048 | 0.216 | 0.100 | 0.063 | 0.062 | 0.416 | 1.43 |
| | RH 2M | Powder | | | | | | | | | | | | | |
| PP00205 | 40° C./75% | White | 98.23 | 4.40 | — | 0.422 | 0.351 | 0.169 | 0.052 | 0.219 | 0.090 | 0.066 | 0.065 | 0.422 | 1.44 |
| | RH 2M | Powder | | | | | | | | | | | | | |
| PP00205 | 5° C. 3M | White | 98.20 | 4.69 | — | 0.416 | 0.436 | 0.155 | — | 0.203 | 0.093 | 0.061 | 0.065 | 0.436 | 1.43 |
| | | Powder | | | | | | | | | | | | | |
| PP00205 | 25° C./60% | White | 98.11 | 4.65 | — | 0.437 | 0.372 | 0.164 | — | 0.225 | 0.094 | 0.062 | 0.065 | 0.437 | 1.42 |
| | RH 3M | Powder | | | | | | | | | | | | | |
| PP00205 | 30° C./65% | White | 98.21 | 4.82 | — | 0.432 | 0.37 | 0.168 | — | 0.228 | 0.096 | 0.057 | 0.064 | 0.432 | 1.43 |
| | RH 3M | Powder | | | | | | | | | | | | | |
| PP00205 | 40° C./75% | White | 98.2 | 4.75 | — | 0.423 | 0.360 | 0.174 | — | 0.218 | 0.102 | 0.065 | 0.064 | 0.423 | 1.41 |
| | RH 3M | Powder | | | | | | | | | | | | | |

| | | | | | | | | | | | | | | | High- | |
|---|---|---|---|---|---|---|---|---|---|---|---|---|---|---|---|---|
| | | | Assay | Water | | | | | | | | | | | est | Total |
| | | Descrip- | (% | (% | RRT | RRT | RRT | RRT | RRT | RRT | RRT | RRT | RRT | RRT | Impu- | Impu- |
| Batch # | Condition | tion | W/W) | W/W | 0.138 | 0.935 | 1.045 | 1.095 | 1.180 | 1.449 | 1.596 | 1.640 | 1.698 | 1.826 | rity | rity |
| PP00205 | 5° C. 4M | White | 98.22 | 5.08 | 0.219 | 0.381 | 0.324 | 0.128 | — | 0.220 | 0.077 | 0.081 | 0.057 | — | 0.381 | 1.487 |
| | | Powder | | | | | | | | | | | | | | |
| PP00205 | 5° C. 5M | White | 98.53 | 4.79 | 0.231 | 0.410 | 0.321 | 0.140 | — | 0.198 | 0.075 | 0.049 | 0.058 | — | 0.410 | 1.482 |
| | | Powder | | | | | | | | | | | | | | |
| PP00205 | 5° C. 6M | White | 98.15 | 4.78 | 0.256 | 0.368 | 0.315 | 0.165 | — | 0.211 | 0.081 | 0.049 | 0.050 | — | 0.368 | 1.495 |
| | | Powder | | | | | | | | | | | | | | |
| PP00205 | 25° C./60% | White | 98.10 | 4.96 | — | 0.476 | 0.324 | 0.191 | — | 0.196 | 0.071 | 0.061 | 0.061 | — | 0.476 | 1.380 |
| | RH 6M | Powder | | | | | | | | | | | | | | |
| PP00205 | 30° C./65% | White | 98.06 | 4.87 | — | 0.418 | 0.352 | 0.134 | — | 0.224 | 0.095 | 0.077 | 0.052 | — | 0.418 | 1.352 |
| | RH 6M | Powder | | | | | | | | | | | | | | |
| PP00205 | 40° C./75% | White | 98.16 | 5.44 | 0.107 | 0.418 | 0.347 | 0.161 | — | 0.187 | 0.087 | 0.050 | 0.052 | — | 0.418 | 1.409 |
| | RH 6M | Powder | | | | | | | | | | | | | | |
| PP00205 | 5° C. 9M | White | 98.14 | 5.39 | 0.222 | 0.390 | 0.372 | 0.142 | — | 0.174 | 0.088 | 0.049 | 0.053 | — | 0.390 | 1.490 |
| | | Powder | | | | | | | | | | | | | | |
| PP00205 | 25° C./60% | White | 97.95 | 5.36 | — | 0.408 | 0.361 | 0.187 | — | 0.225 | 0.096 | 0.062 | 0.055 | 0.053 | 0.408 | 1.477 |
| | RH 9M | Powder | | | | | | | | | | | | | | |
| PP00205 | 30° C./65% | White | 97.89 | 4.91 | — | 0.407 | 0.340 | 0.150 | — | 0.216 | 0.103 | 0.064 | 0.053 | — | 0.407 | 1.333 |
| | RH 9M | Powder | | | | | | | | | | | | | | |
| PP00205 | 5° C. 12M | White | 98.31 | 5.97 | — | 0.438 | 0.383 | 0.177 | — | 0.217 | 0.090 | 0.066 | 0.066 | — | 0.438 | 1.437 |
| | | Powder | | | | | | | | | | | | | | |
| PP00205 | 25° C./60% | White | 98.10 | 5.57 | 0.204 | 0.399 | 0.344 | 0.165 | — | 0.195 | 0.086 | 0.052 | 0.061 | — | 0.399 | 1.506 |
| | RH 12M | Powder | | | | | | | | | | | | | | |
| PP00205 | 30° C./65% | White | 97.80 | 5.33 | — | 0.424 | 0.363 | 0.172 | — | 0.197 | 0.089 | 0.064 | 0.058 | — | 0.424 | 1.367 |
| | RH 12M | Powder | | | | | | | | | | | | | | |

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, that has X-ray powder diffraction peaks at about 5.43, 7.95, 9.61, 11.29, 11.92, 18.91, 19.25, 22.78, and 23.95 2θ°.

2. The crystalline polymorph of claim 1 that has X-ray powder diffraction peaks at about 3.99, 5.43, 5.74, 7.95, 9.61, 11.29, 11.92, 15.91, 18.91, 19.25, 22.78, 23.95, and 28.02° 2θ°.

3. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by a differential scanning calorimetry curve that exhibits an endotherm with an extrapolated onset temperature of about 176.43° C. and associated heat of about 13.55 J/gram.

4. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by an infrared spectrum having IR bands at 3301, 2964, 2871, 1902, 1646, 1314, 1225, 1157, 845, 699, 618 and 522 cm⁻¹.

5. The crystalline polymorph of claim 4, wherein the infrared spectrum is substantially as shown in FIG. 9.

6. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, that has X-ray powder diffraction peaks at about 3.76, 6.08, 7.19, 8.90, 12.30, 12.86, 17.62, 20.16, 24.41, 26.59 and 28.77 2θ°.

7. The crystalline polymorph of claim 6 that has X-ray powder diffraction peaks at about 3.76, 5.32, 6.08, 7.19, 8.90, 9.34, 11.27, 12.30, 12.86, 15.29, 16.18, 17.62, 20.16, 21.08, 21.51, 22.57, 24.41, 24.63, 25.15, 26.59, 28.77, 35.67, 37.48° 2θ°.

8. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by a differential scanning calorimetry curve that exhibits an endotherm with an extrapolated onset temperature of about 187° C. and associated heat of about 21.64 J/gram.

9. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by an infrared spectrum having IR bands at 3398, 2929, 2364, 1738, 1703, 1656, 1596, 1561, 1511, 1314, 1225, 1117, 843, 752 and 700 cm⁻¹.

10. The crystalline polymorph of claim 9, wherein the infrared spectrum is substantially as shown in FIG. 10.

11. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, that has X-ray powder diffraction peaks at about 4.72, 7.01, 9.38, 13.59, 18.28, 19.56, 20.48, 22.33, 22.97, 23.51 and 27.29° 2θ°.

12. The crystalline polymorph of claim 11 that has X-ray powder diffraction peaks at about 3.71, 4.72, 7.01, 7.35, 9.38, 10.16, 13.06, 13.59, 14.03, 14.57, 15.85, 17.09, 17.64, 18.28, 19.56, 20.48, 22.33, 22.97, 23.51, 27.29° 2θ°

13. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by a differential scanning calorimetry curve that exhibits an endotherm with an extrapolated onset temperature of about 178.49° C. and associated heat of about 18.14 J/gram.

14. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by an infrared spectrum having IR bands at 3402, 2966, 1655, 1560, 1514, 1222, 1156, 1110, 1031, 844 and 700 cm⁻¹.

15. The crystalline polymorph of claim 14, wherein the infrared spectrum is substantially as shown in FIG. 11.

16. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, that has X-ray powder diffraction peaks at about 5.72, 9.42, 10.16, 10.42, 11.40, 18.56, 19.48, 21.03 and 21.83 2θ°.

17. The crystalline polymorph of claim 17 that has X-ray powder diffraction peaks at about 4.09, 5.72, 9.42, 10.16, 10.42, 11.40, 11.80, 14.99, 17.39, 18.56, 19.48, 21.03, 21.83, 22.83°2θ°.

18. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by a differential scanning calorimetry curve that exhibits an endotherm with an extrapolated onset temperature of about 179° C. and associated heat of about 11.23 J/gram.

19. A crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, characterized by an infrared spectrum having IR bands at 3400, 2965, 2343, 1650, 1563, 1409, 1013 and 619 cm⁻¹.

20. The crystalline polymorph of claim 19, wherein the infrared spectrum is substantially as shown in FIG. 12.

21. A pharmaceutical composition comprising the crystalline polymorph of any one of claims 1-20.

22. The pharmaceutical composition of claim 21, further comprising a pharmaceutically acceptable diluent, excipient, carrier or mixture thereof.

23. The pharmaceutical composition of claim 21, wherein the composition is formulated as a film-coated tablet.

24. A method of treating a disease selected from the group consisting of cholesterol-related disease, diabetes, diabetes-related disease, cerebrovascular disease and cardiovascular disease in a patient comprising administering to a patient having or at risk of having such disease a therapeutically effective amount of the pharmaceutical composition of claim 21.

25. The method of claim 24, wherein the disease is a cholesterol-related disease selected from the group consisting of arteriosclerosis, atherosclerosis, hypercholesterolemia, hyperlipidemia, hyperlipoproteinemia, hypertriglyceridemia, hypertension, stroke, ischemia, endothelium dysfunction, peripheral vascular disease, peripheral arterial disease, coronary heart disease, myocardial infarction, cerebral infarction, myocardial microvascular disease, dementia, Alzheimer's disease, osteoporosis, osteopenia, angina, restenosis and combinations thereof.

26. A method of making a crystalline polymorph form of a HMG-CoA reductase inhibitor comprising:
a. dissolving (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, in a solvent comprising water and ethyl acetate to form a solution;
b. cooling the solution to less than about 30° C.; and,
c. removing the solvent from the solution to recover a Form I crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt.

27. A method of making a crystalline polymorph form of a HMG-CoA reductase inhibitor comprising:
a. dissolving (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, in a solvent comprising water and ethanol to form a solution;
b. cooling the solution to less than about 30° C.; and,
c. removing the solvent from the solution to recover a Form I crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt.

28. A method of making a crystalline polymorph form of a HMG-CoA reductase inhibitor comprising:
a. dissolving (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, in a solvent comprising water and acetonitrile to form a solution;
b. cooling the solution to less than about 30° C.; and,
c. removing the solvent from the solution to recover a Form II crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt.

29. A method of making a crystalline polymorph form of a HMG-CoA reductase inhibitor comprising:
a. dissolving (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, in water to form a solution;
b. cooling the solution to less than about 30° C.; and,
c. removing the water from the solution to recover a Form III crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt.

30. A method of making a crystalline polymorph form of a HMG-CoA reductase inhibitor comprising:
a. dissolving (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt, in a solvent comprising water and acetone to form a solution;
b. cooling the solution to less than about 30° C.; and,
c. removing the solvent from the solution to recover a Form IV crystalline polymorph of (3R, 5R)-7-[2-(4-fluorophenyl)-5-isopropyl-3-phenyl-4-[(4-hydroxymethylphenylamino)carbonyl]-pyrrol-1-yl]-3,5-dihydroxy-heptanoic acid, hemi calcium salt.

31. The method of any one of claims 26-30, further comprising drying the recovered crystalline polymorph.
